(19) United States
(12) Patent Application Publication
(10) Pub. No.: US 2006/0166210 A1
(43) Pub. Date: Jul. 27, 2006

(54) Methodology of estimating the conformation of a protein by proteolysis

(76) Inventors: Linda Fryklund, Stockholm (SE); Mark Lewis, Cardiff (GB); David Cooper, Cardiff (GB)

Correspondence Address: KING & SCHICKLI, PLLC, 247 NORTH BROADWAY, LEXINGTON, KY, 40507, US

(21) Appl. No.: 10/535,013

(22) PCT Filed: Nov. 4, 2003
(86) PCT No.: PCT/GB03/04740

§ 371 Date: Mar. 13, 2006

(30) Foreign Application Priority Data
Nov. 12, 2002 (GB) 0226441.4
Apr. 10, 2003 (GB) 0308242.7
Apr. 10, 2003 (GB) 0308238.5
Nov. 12, 2002 (GB) 02/005112

(51) Int. Cl. C12Q 1/68; C12Q 1/37

(52) U.S. Cl. 435/6; 435/23

(57) ABSTRACT

The invention relates to a method for determining the effect of a polymorphism or mutation on the structural properties of a protein wherein the method relies on the structural properties of the protein and so its cleavage during proteolysis.

The invention relates to a novel method for determining the significance of polymorphisms or mutations in a nucleic acid molecule encoding a protein.

Since the advent of gene sequencing technology in the late 1980's and the establishment of the human genome project in 1990 an enormous amount of information has been discovered about the sequence, or nature, of each gene in the human genome. Moreover, as the human genome project has developed the methods used to sequence genes have evolved considerably and this has led to the detection of variations within genes. Given that a typical gene could be 30 kilobases in length and that variations occur on average every 1100 bases, it follows that a tremendous amount of work needs to be undertaken in order to determine which variants are of clinical or technological significance. However, this is a prerequisite step if one is to exploit the knowledge available in the human genome project and so be in a position to understand, for example, the human condition, and particularly human diseases, and factors that may influence same and so lead to new therapies.

Typically, investigators in the field of human genetics who have obtained the sequence of the normal, or wild-type gene, set about looking for significant changes in the gene by sequencing nucleic acid molecules from individuals who are thought to harbour a gene variant. Such individuals are people exhibiting the symptoms of a specific disease which is thought to be related to the dysfunction of a particular gene. Once a gene variant has been sequenced and compared with the wild-type further investigations are then undertaken to examine the cell biology of the protein encoded by the variant gene. The results of these investigations are then examined in the light of the physical symptoms in order to deduce a correlation.

It therefore follows that unravelling the nature of a gene variant and relating it to function and then clinical symptoms is a long and tedious process, especially when one considers that a given gene can be 3.6% polymorphic. It is therefore apparent that simply identifying which variant to investigate further can be a difficult step in itself. This is true not only for the field of human genetics but also in respect of studies of other animal and plant species.

With this in mind, we have developed a novel assay for quickly and efficiently determining the likely significance of a gene variant.

Our novel methodology is based upon the basic structure of proteins.

The basic structural unit of a protein is an amino acid. An amino acid consists of an amino group, a carboxyl group, a hydrogen atom and a distinctive R group bonded to a carbon atom, conventionally known as the side chain. There are 22 amino acids and any number and combination of them are able to join, via peptide bonds, to form a sequence, or chain, of amino acids known as peptides. Thus the sequence of bonds running the length of the peptide chain is known as the backbone. Additionally, intra and inter peptide chain linkages also exist, for example, in the former instance the amino group of lysine can form a peptide bond with the gamma carboxyl group of glutamic acid; and, in the latter instance, bonds may also exist between side chains of amino acids as a result of the formation of disulphide bonds thus forming crosslinks between separate peptide chains. Adjacent peptide chains can therefore join to form a secondary structure such as dimers or trimers etc. The secondary structures can then fold, due to the nature of the interaction of adjacent amino acids, to form a three dimensional tertiary structure. This tertiary structure represents the active form of the protein and may comprise sites, or pockets, into which other molecules fit in order to activate the protein or allow the protein to respond thereto.

Digestion, or break down, of proteins in a controlled fashion, occurs all the time during the process of alimentary digestion. A class of enzymes known as proteases perform this function. They basically attack specific bonds in order to cleave the protein at sites where these bonds exist. It follows that different proteins will have different susceptibilities to various enzymes depending upon their primary structure.

Whilst all this information is known, no-one has thought to take advantage of it before in relation to genetics and, in particular, in relation to screening a number of genetic variants whose functional, or even clinical, significance is unknown. Accordingly, no-one has thought to use this information as a basis to tackle the large number of genetic variants that exist in order to determine which are the clinically, or technologically, significant variants.

However, we have used this information to develop a novel assay which can screen any number of variants, simultaneously if required, in order to determine which, if any, require further investigation.

Our methodology is quick, efficient and inexpensive to perform.

### STATEMENTS OF INVENTION

According to the invention there is therefore provided a method for determining the significance of a given nucleic acid polymorphism or mutation, in a nucleic acid molecule, on the structural properties of a protein encoded by said nucleic acid molecule comprising:
(a) exposing the protein encoded by said nucleic acid molecule to at least one protease; and
(b) determining whether, or to what extent, proteolytic cleavage takes place;

and, optionally,
(c) comparing this proteolytic cleavage with that of the wild-type protein when exposed to the same protease(s).

According to a further aspect of the invention there is provided a screening method for determining the significance of a plurality of variants of at least one gene comprising:
(a) obtaining a sample of protein encoded by each of said variants;
(b) exposing each protein to at least one protease;
(c) determining whether, or to what extent, proteolytic cleavage takes place; and
(d) comparing this proteolytic cleavage with that of the wild-type protein when exposed to the same protease(s).

In a preferred embodiment of the invention when the above screening method is employed, the plurality of protein variants are exposed to a plurality of proteases and the corresponding proteolytic cleavage is determined. Most ideally, the screening methodology involves examining the plurality of variants relating to different genes. Thus, in a single batch, the plurality of variants corresponding to multiple genes are examined in respect of at least one protease, and ideally in respect of a plurality of proteases and a determination of proteolytic cleavage is made in respect of the digestion of each variant by each protease.

It will be apparent to those skilled in the art that, using this methodology, where a plurality of proteases are employed a cleavage or digestion profile will be provided in respect of each variant and this parameters ideally can be compared with the digestion profile of the wild-type protein and so used to determine the functional significance of a variant of any one or more of the said genes.

In a preferred embodiment of the invention said protein encoded by said nucleic acid molecule or gene variant is exposed to a plurality of proteases and ideally different proteases which attack different bonds. Proteases that are suitable for use in the methodology of the invention include: Trypsin, chymotrypsin, proteinase K, aminopeptidase, carboxypeptidase, collagenase, elastase, Kallikrein, metalloendopeptidase, papain, pepsin, and indeed any other known protease.

Notably, where cleavage is different from that exhibited by the wild-type, one would conclude that the variant, or indeed a combination of variants, was significant. This is because the variant(s) would either render the protein more vulnerable to digestion or confer resistance to digestion as a result of alteration(s) to the tertiary, or structural, form of the protein.

In yet a further preferred embodiment of the invention a plurality of proteins encoded by a plurality of genetic variants are tested in parallel and thus the methodology of the invention may be performed as a screening methodology where a plurality of incubation receptacles are filled with a corresponding plurality of proteins to be tested and then said proteins are exposed to a selected protease, or group of proteases, or vice versa, either simultaneously or successively.

More preferably still, the methodology of the invention involves incubating the protein(s) to be tested with the said protease(s) under conditions that support the activity of the relevant enzyme(s). For example, this may involve exposing the test protein to the enzyme at a temperature at which the enzyme is optimally functional, such as 37° C., and for a time sufficient for the enzyme to perform its activity, for example between 15 minutes and 1.5 hours.

More preferably still, after a suitable length of time the incubation period is terminated, for example, by adding an enzyme inhibitor to the incubation receptacle. Finally, proteolytic cleavage is assessed using any conventional protein assay technique such as, for example, SDS-PAGE analysis either followed by staining the gel (coomassie blue or silver staining) or by western blotting. Optionally, additional studies may then be undertaken to determine the functionality of the protein variant.

In yet a further preferred embodiment of the invention the technique undertaken, in order to determine the extent of proteolytic cleavage, involves assaying not only each test protein but also the wild-type protein that, ideally, has been exposed to the relevant enzyme(s) and, ideally also, a sample of the wild-type and test protein(s) that has not been exposed to the relevant enzyme(s). In this way, a positive and a negative control are included in the assay for the purpose of determining the amount of proteolytic cleavage that the test protein exhibits vis a vis the wild-type protein and also the background level of protein degradation experienced as a result of the assay conditions.

It is to be understood that the invention is not to be limited to the specific assay that is chosen to assess proteolytic cleavage, rather the invention, principally, lies in the use of the technique of proteolytic cleavage to assay the likely functional significance of genetic variants.

It follows from the information above regarding the tertiary structure of the protein that the nature of the amino acids in the peptide chain will determine the protein folding and so susceptibility to different enzymes. In turn, the nature of the amino acids in the peptide chain will be determined by the nucleic acid coding sequence and so variations in this sequence will lead to variations at the amino acid level and so differential protein folding and thus variable susceptibility to proteolytic cleavage.

Given that the assay is quick and efficient to perform, a whole range of proteins, each coded by a genetic variant for a given gene, or more than one gene, can be simultaneously assayed in order to determine which variant gives rise to a change in the tertiary structure of the amino acid and thus which is most likely to affect the functioning of the protein.

An embodiment of the invention will now be described by way of example only, with reference to variants in the growth hormone gene (GH1) and the following examples.

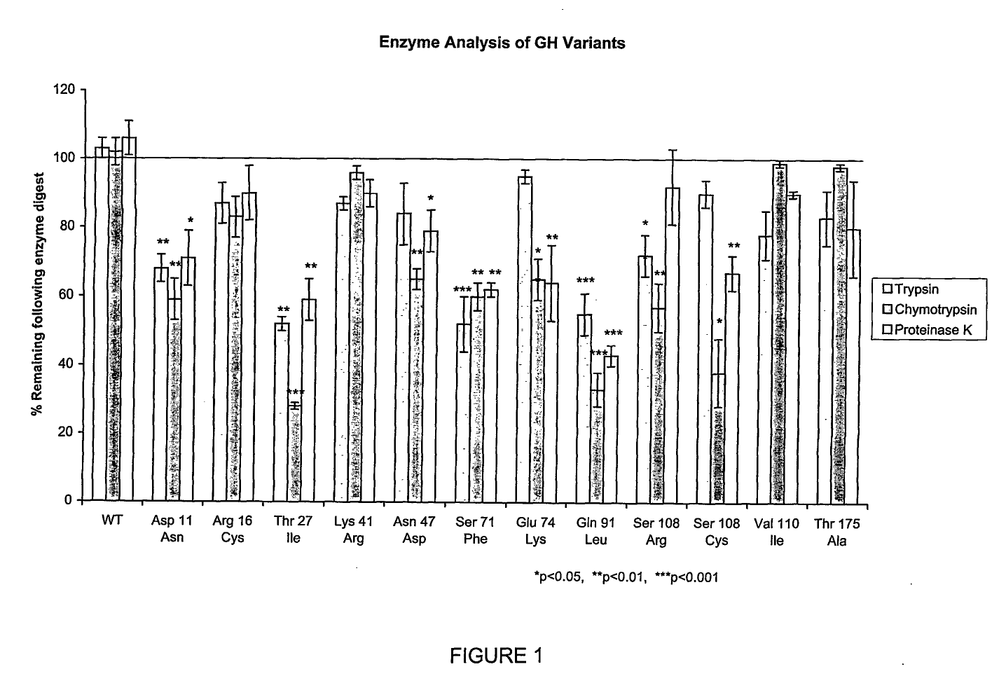

In FIG. 1 there is shown the digestion profile of a number of variants of the growth hormone gene when exposed to trypsin, chymotrypsin or proteinase K.

### EXPERIMENTAL SUBJECTS

The experimental subjects in which the mutations for the proteolysis study were identified are those described in the original Human Mutation paper, Millar et al.

Two different patient groups were studied. The first comprised 41 unrelated children of Caucasian origin with short stature (age 1-15 years) who matched the specific selection criteria applied in Cardiff (the “Cardiff criteria”) outlined below. Details were taken of family history, clinical and auxological variables, and previously performed laboratory investigations (Table 1). Standard deviation scores (SDS) were calculated for birth weight, height prior to GH treatment, body mass index prior to GH treatment, height velocity immediately prior to GH secretion testing, paternal and maternal heights, and the target for adult height derived from parental measurements (Table 1). The degree of bone age delay and the results of GH secretion tests were also noted. Blood samples for DNA analysis were taken from the index case and appropriate close relatives.

The second group comprised 11 unrelated patients with short stature and idiopathic isolated growth hormone deficiency (IGHD) in whom GH1 gene deletions had been excluded by Southern blotting. Eight of these individuals came from familities with two or more first-degree relatives with IGHD (familial IGHD) whilst 3 individuals represented sporadic cases of IGHD. In only one of the familial IGHD cases (family 37) did short stature appear to segregate as an autosomal dominant trait. Blood samples for DNA analysis were taken from available relatives in each family.

Control DNA samples were obtained from lymphocytes taken from 154 male British army recruits of Caucasian origin who were unselected for height. Height data were available for 124 of these individuals (mean: 1.76±0.07 m) and the height distribution was found to be normal (Shapiro-Wilk statistic W=0.984, p=0.16). Ethical approval for these studies was obtained from the Multi-Regional Ethics Committee (MREC).

### PATIENT SELECTION CRITERIA

The key criterion for inclusion in this study was that the clinician assessing the child should have had sufficient concern with regard to the child's growth pattern to warrant GH secretion testing. The children selected exhibited a clinical phenotype that adhered to the following criteria, henceforth termed “Cardiff criteria”:
(a) sufficient clinical concern to have warranted GH secretion testing, regardless of the type of test, the test results, or indeed whether the child attended for testing;
(b) no recognisable pathology likely to account for the observed growth failure;
(c) short stature: defined as a predicted height trajectory below the lower limit of an individual's estimated target adult height, based upon the heights of that individual's parents (Tanner and Whitehouse 1976);
(d) height velocity on or below the 25^{th} percentile for age (uncorrected for bone age); and
(e) evidence of bone age delay in those pre-pubertal when compared with chronological age by reference to the Tanner-Whitehouse scale (TW2 method; Tanner et al, 1983). This delay should be of at least two years except in children of ≦5 years of age.

### MATERIALS & METHODS

Polymerase Chain Reaction (PCR) Amplification of a GH1-Specific Fragment

Genomic DNA was extracted from patient lymphocytes by standard procedures. PCR amplification of a 3.2 kb GH1-specific fragment was performed as described (2).

Cloning and Sequencing of GH1 Gene-Specific PCR Fragments

GH1 gene-specific (3.2 kb) PCR fragments were sequenced directly with BigDye v3.0 (Applied Biosystems, Foster City, Calif.) and analysed on an ABI 3100 DNA sequencer (Applied Biosystems) as described (2). Additional primers used for sequencing in the reverse direction were GHBFR (5′ TGGGTGCCCTCTGGCC 3′; −262 to −278), GHSEQ1R (5′ AGATTGGCCAAATACTGG 3′; +215 to +198), GHSEQ2R (5′ GGAATAGACTCTGAGAAAC 3′; +785 to +767), GHSEQ3R (5′ TCCCTTTCTCATTCATTC 3′; +1281 to +1264), GHSEQ4R (5′ CCCGAATAGACCCCGC 3; +1745 to +1730) [Numbering relative to the transcriptional initiation site at +1; GenBank Accession No. J03071]. Samples containing sequence variants were cloned into pGEM-T (Promega, Madison Wis.) followed by sequencing of a minimum of four clones per individual.

In vitro Expression and Assay of Biological Activity of GH Variants

A cloned wild-type GH1 cDNA incorporating a His tag on the carboxy terminal was modified using site-directed mutagenesis as previously described (3) to generate the GH variants.

This vector was then transfected into High Five insect cells (Invitrogen) as previously described (3), and human GH in the culture supernatants quantified by ELISA (DRG Diagnostics, Marburg, Germany). The cross-reactivity in the ELISA of the GH variants and insect cell-expressed wild-type GH was confirmed by dilutional analysis to be equal to that of the assay reference preparation (calibrated against the MRC 1^{st} IRP 80/505 reference preparation).

Proteolytic Digestion of the GH Variants

Trypsin, chymotrypsin, or proteinase K (all Sigma, Poole, UK) were added to a final concentration of 0.1 μg/ml to 100 μl culture medium harvested from insect cells expressing either wild-type GH or a variant (60 nM) and then incubated at 37° C. for 1 hr. Previous dose-dependent studies on wild-type GH had shown that 0.1 μg/ml was the lowest concentration at which GH degradation was detectable by all three enzymes. After the 1 hr treatment period, 10 μl trypsin-chymotrypsin inhibitor (500 μg/ml) was added to stop the trypsin and chymotrypsin digests and 1 μl PMSF (0.1M) was added to stop the proteinase K digest. Each reaction was then incubated for a further 15 mins at 37° C. Samples were analysed by SDS-PAGE on a 12% gel using a mini gel apparatus (Bio-Rad Laboratories, Hercules, Calif.). Equivalent amounts of undigested wild-type GH and variant that had been incubated for 1 hour at 37° C. were also run on the gel. The gel was electroblotted onto PVDF membrane as previously described (6), probed with a mouse monoclonal anti-human GH antibody (Lab Vision, Fremont, Calif.), diluted 1:500, detected using an anti-mouse IgG-horse radish peroxidaseHRP (HRP) conjugate (1:5000, Amersham Biosciences) and visualised by enhanced chemiluminescence (ECL Plus, Amersham Biosciences). Films were analysed using the Alpha Imager 1200 digital imaging system (Alpha Innotech Corp, San Leandro, Calif.) and the results expressed as the amount of GH remaining following enzyme digestion as a percentage of undigested GH. The experiments were repeated 3 times and assessed statistically by a two-tailed t-test.

Molecular Modelling

The variants were structurally analysed by inspection of the appropriate variant amino acid residue in the X-ray crystallographic structure of human GH (PDB: 3HHR) [8]. The wild-type and mutant GH structures were compared with respect to electrostatic interactions, hydrogen bonding, hydrophobic interactions and surface exposure. Molecular graphics were performed using the ICM molecular modelling software suite (Molsoft LLC, San Diego, Calif.).

### RESULTS

Proteolysis Studies

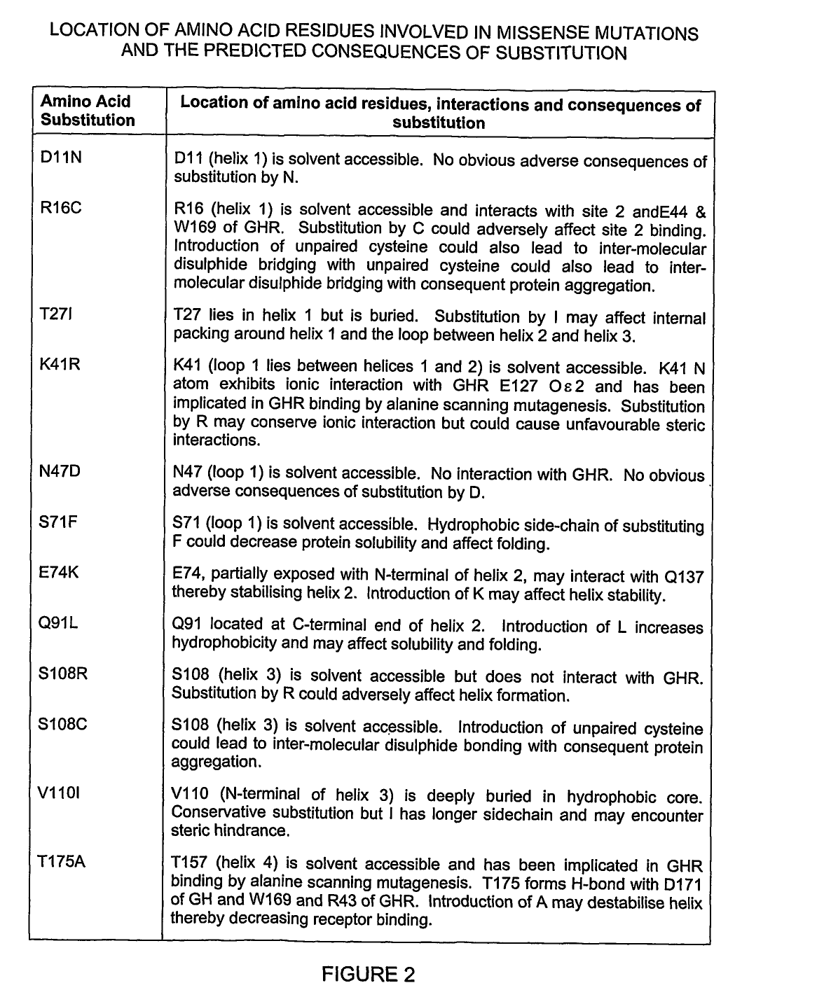

FIG. 1 shows the results of enzyme analysis performed on a number of GH variants in order to determine which, if any, of these variants alter the structural properties of the protein and so are likely to interfere with the activity thereof. Twelve variants were examined and it can be seen that with respect to the wild-type (WT), left hand side of the FIGURE, the majority of these variants have an effect on the susceptibility of the protein to proteolytic digestion. The variants Thr27Ile and Gln91Leu were particularly vulnerable to proteolysis and, in each case, proteolysis proceeded most efficiently using the enzyme chymotrypsin. With reference to FIG. 2 it can be seen that the variant Thr27Ile is predicted to affect internal packing around its helix 1 and the loop between helix 2 and helix 3. This obviously has important structural implications which is reflected in the data shown in FIG. 1. Similarly, in the case of Gln91Leu the substitution increases hydrophobicity and may affect solubility and folding. This has implications for the structure of the protein and thus its susceptibility to proteolysis.

In contrast, Arg16Cys and Lys41Arg, whilst showing different proteolysis profiles, compared to the wild-type, are less affected than the previous variants. However, for Arg16Cys the predicted structural changes concern inter-molecular bridging rather than adverse effects on the shape of the protein. This could explain why the proteolysis profile is less affected. In the case of Lys41Arg, the variant is thought to conserve ionic interactions but may lead to steric hindrance. Again, the implication here is that the shape of the protein is likely to be conserved.

In contrast, other variants showed only marginal susceptibility to proteolysis such as VAL110Ile and Thr175Ala which were both most resistant to proteolysis by the enzyme chymotrypsin.

The results of this study show that GH variants can be characterised in terms of their proteolysis signature in response to selected proteases and this information represents a first step towards selecting clinically and technologically important variants for further analysis.

TABLE 1
Children with short stature adhering to the specific (“Cardiff”) selection
criteria; clinical and auxological variables, and laboratory investigations.

| P | S | BW | GH | T | H | HA | BA | BMI | HV | Mat | Pat |
|---|---|---|---|---|---|---|---|---|---|---|---|
| 10 | M | −2.4 | 38.7 | E | −1.8 | 8.9 | 7.0 | −1.4 | −0.9 | 0.9 | −2.6 |
| 12 | F | −2.7 | X | X | −1.8 | 1.0 | 0.5 | −3.6 | −4.3 | −1.5 | −0.5 |
| 20 | M | 2.1 | 4.1 | I | −3.4 | 7.3 | 5.3 | 1.0 | −1.8 | −1.8 | 0.1 |
| 33* | M | −0.4 | X | X | −2.1 | 6.5 | 3.9 | −0.7 | −1.5 | −1.0 | −0.2 |
| 53 | M | −3.2 | 27.2 | C | −3.8 | 13.5 | 11.5 | −4.9 | −1.3 | −2.8 | −1.5 |
| 57 | F | −3.4 | 27.3 | R | −2.8 | 2.7 | 1.5 | −1.7 | −1.7 | 0.3 | 0.7 |
| 62 | F | −1.9 | 27.0 | G | −4.4 | 4.6 | 3.5 | 0.6 | −0.7 | −1.5 | −1.9 |
| 63 | M | −0.9 | 1.3 | G | −4.5 | 8.2 | 6.2 | −0.2 | −4.4 | 0.6 | −1.1 |
| 66 | M | −1.0 | 18.8 | I | −2.9 | 8.1 | 6.1 | −0.3 | −1.2 | −3.9 | −1.6 |
| 71§ | F | −2.9 | 4.0 | I | −1.4 | 3.7 | 3.7 | −0.7 | −0.5 | −2.4 | −2.4 |
| 75 | F | 0.2 | 6.8 | C | −6.2 | 14.1 | 13.5 | 4.2 | −3.5 | −0.6 | 0.3 |
| 76§ | F | −1.8 | 18.3 | I | −2.2 | 8.0 | 7.0 | −1.3 | −1.8 | −2.4 | −2.2 |
| 79 | M | −3.6 | 28.8 | C | −5.3 | 3.3 | 1.0 | −1.2 | −1.1 | −1.9 | −1.1 |
| 83 | M | −2.8 | N | R | −3.5 | 2.0 | 1.0 | −1.3 | −2.1 | 2.1 | 0.7 |
| 1 | F | 1.4 | 48.6 | I | −3.9 | 13.1 | 11.1 | −0.9 | 0.1 | −3.2 | −1.6 |
| 2 | F | −1.4 | 20.2 | I | −3.0 | 15.1 | 13.1 | 1.1 | −1.0 | −0.1 | −1.7 |
| 3 | F | −1.2 | 3.7 | C | −3.9 | 2.8 | 1.0 | 2.3 | −2.2 | −0.8 | −1.1 |
| 4 | M | 0.3 | 26.7 | I | −3.4 | 7.5 | 4.7 | 1.4 | −2.2 | −1.7 | −1.2 |
| 5 | M | −1.7 | 7.7 | I | −3.0 | 9.0 | 6.0 | 1.1 | −0.8 | −1.5 | −0.2 |
| 6 | M | −3.1 | 28.4 | C | −4.2 | 5.8 | 3.0 | −0.9 | −1.5 | −1.9 | −3.7 |
| 7 | F | −2.7 | 111.3 | C | −3.0 | 5.6 | 2.5 | −2.8 | 0.4 | 0.8 | −1.2 |
| 9 | F | 0.6 | 9.8 | I | −2.0 | 13.3 | 11.3 | 2.6 | 1.6 | −0.8 | −1.0 |
| 11 | M | 0.3 | 0 | C | −4.8 | 5.8 | 3.4 | −0.1 | −1.7 | −4.1 | −1.4 |
| 13 | F | −3.1 | 16.0 | C | −4.7 | 4.3 | 2.5 | −1.4 | −4.0 | 0.2 | −1.3 |
| 14 | M | −0.5 | 13.2 | C | −1.9 | 12.8 | 8.8 | −1.2 | −0.7 | −0.2 | −1.0 |
| 15 | F | −0.9 | 0 | C | −5.0 | 5.0 | 2.0 | 0.8 | −5.0 | −0.2 | −0.8 |
| 16 | M | −0.5 | 4.6 | I | −3.1 | 6.8 | 4.8 | −1.0 | −2.1 | −0.3 | −0.6 |
| 18 | M | −3.8 | 18.0 | I | −4.9 | 8.8 | 4.8 | −1.2 | −2.3 | −1.1 | −0.8 |
| 19 | F | −0.5 | 2.1 | I | −2.2 | 4.3 | 2.0 | −0.1 | −3.0 | −0.5 | 0.2 |
| 26 | M | −0.2 | 38.6 | C | −2.8 | 12.0 | 9.0 | −2.1 | −0.1 | −0.2 | −1.0 |
| 27 | F | −1.4 | 5.6 | I | −3.9 | 13.4 | 10.5 | 0.2 | 0.2 | −2.1 | −1.8 |
| 34 | F | −5.4 | 10.0 | R | −3.8 | 1.4 | 1.0 | −2.6 | −0.8 | −0.1 | −0.0 |
| 40 | M | −1.4 | X | X | −1.4 | 12.8 | 10.2 | −0.4 | −1.2 | 1.0 | −0.8 |
| 58 | M | −3.8 | <1 | I | −4.5 | 8.8 | 6.8 | −2.4 | −6.6 | −3.8 | −1.6 |
| 59 | F | −0.1 | <1 | I | −3.6 | 8.0 | 5.7 | −2.6 | −0.9 | −1.5 | −0.8 |
| 68 | M | −0.9 | X | X | −3.1 | 8.0 | 5.0 | −0.1 | −1.2 | −1.1 | −1.2 |
| 77 | M | −0.6 | 48.0 | C | −3.9 | 5.7 | 2.8 | −0.8 | −1.6 | −0.3 | −1.3 |
| 80 | M | −3.6 | 33.9 | C | −4.2 | 3.5 | 1.3 | −3.0 | −0.8 | −0.6 | −0.5 |
| 81 | M | −1.8 | N | R | −3.4 | 5.0 | 2.3 | −3.1 | −2.3 | −0.1 | −0.5 |
| 82 | F | −1.9 | 24.2 | C | −6.8 | 2.0 | 1.0 | 0.3 | −2.6 | −0.3 | 0.4 |
| 84 | F | −0.3 | 45.0 | C | −4.0 | 5.8 | 3.5 | −0.1 | −3.2 | −1.5 | 0.8 |

Key.
P: patient number;
S: sex;
BW: birth weight standard deviation score (SDS);
GH: GH secretion test result (mlU/L);
N: “normal”,
T: GH secretion test type:
I: insulin tolerance test,
C: clonidine,
G: glucagon,
E: exercise,
R: random,
X: test declined.
H: height SDS;
HA: age at height SDS and bone age assessment;
BA: bone age in years;
BMI: body mass index SDS;
HV: height velocity SDS;
Mat: maternal height SDS;
Pat: paternal height SDS.
Data from patients possessing GH1 gene lesions are shown in bold type.
*one of two siblings with a similar phenotype.
§family history of GHD.

### REFERENCES

1 Ranke M B 1996 Towards a consensus on the definition of idiopathic short stature. Horm Res 45 Suppl. 2:64-66
2 Horan M, Millar D S, Hedderich J, Lewis G, Newsway V, Mo N, Fryklund L, Procter A M, Krawczak M, Cooper D N 2003 Human growth hormone 1 (GH1) gene expression: complex haplotype-dependent influence of polymorphic variation in the proximal promoter and locus control region. Hum Mutat 21:408-423
3 Millar D S, Lewis M D, Horan M, Newsway V, Easter T E, Gregory J W, Fryklund L, Norin M, Crowne E C, Davies S, Edwards P, Kirk J, Waldran K, Smith P J, Phillips III J A, Scanlon M F, Krawczak M, Cooper D N, Procter A M 2003 Novel mutations of the growth hormone 1 (GH1) gene disclosed by modulation of the clinical selection criteria for individuals with short stature. Hum Mutat 21:424-440
4 Ross R J, Esposito N, Shen X Y, von Laue S, Chew S L, Dobson P R, Postel-Vinay M C, Finidori J 1997 A short isoform of the human growth hormone receptor functions as a dominant negative inhibitor of the full-length receptor and generates large amounts of binding protein. Mol Endocrinol 11:265-273
5 von Laue S, Finidori J, Maamra M, Shen X-Y, Justice S, Dobson PRM, Ross RJM 2000 Stimulation of endogenous GH and interleukin-6 receptors selectively activates different Jaks and Stats, with a Stat5-specific synergistic effect of dexamethasone. J Endocrinol 165:301-311
6 Lewis M D, Ham J, Rees D A, Lewis B M Scanlon M F 2002 Mitogen-activated protein kinase mediates epidermal growth factor-induced morphogenesis in pituitary GH₃ cells. J Neuroendocrinol 14:361-367
7 Green H, Kehinde O 1976 Spontaneous heritable changes leading to increased adipose conversion in 3T3 cells. Cell 7:105-113
8 de Vos A M, Ultsch M, Kossiakoff M 1992 Human growth hormone and extracellular domain of its receptor: crystal structure of the complex. Science 255:306-312
9 Krawczak M, Chuzhanova N A, Cooper D N 1999 Evolution of the proximal promoter region of the mammalian growth hormone gene. Gene 237:143-151
10 Clackson T, Wells J A 1995 A hot spot of binding energy in the hormone-receptor interface. Science 267:383-386
11 Clackson T, Ultsch M H, Wells J A, de Vos AM 1998 Structural and functional analysis of the 1:1 growth hormone:receptor complex reveals the molecular basis for receptor affinity. J Mol Biol 277:1111-1128
12 Cunningham B C, Wells J A 1989 High-resolution epitope mapping of hGH-receptor interactions by alanine-scanning mutagenesis. Science 244:1081-1084
13 Hansen J H, Wang X, Kopchick J J, Bouchelouche P, Neilson J H, Galsgaard E D, Billestrup N 1996 Identification of tyrosine residues in the intracellular domain of the growth hormone receptor required for transcriptional signalling and STAT 5 activation. J Biol Chem 271:12669-12673
14 Sotiropoulos A, Perrot-Applanat M, Dinerstein H, Pallier A, Postel-Vinay M-C, Finidori J, Kelly PA 1994 Distinct cytoplasmic regions of the growth hormone receptor are required for activation of JAK2, mitogen-activated protein kinase, and transcription. Endocrinology 135:1292-1298
15 VanderKuur J, Allevato G, Billestrup N, Norstedt G, Carter-Su C 1995 Growth hormonr-promoted tyrosyl phosphorylation of SHC proteins and SHC association with Grb2. J Biol Chem 270:7587-7593
16 VanderKuur J A, Butch E R, Waters S B, Pessin J E, Guan K L, Carter-Su 1997 Signalling molecules involved in coupling growth hormone receptor to mitogen-activated protein kinase activation. Endocrinology 138:4301-4307
17 Liang L, Jiang J, Frank S J 2000 Insulin receptor substrate-1 enhancement of growth hormonr-induced MAP kinase activation. Endocrinology 141:3328-3336
18 Kim S-O, Loesch K, Wang X, Jiang J, Mei L, Cunnick J M, Wu J, Frank S J 2002 A role for Grb2-associated binder-1 in growth hormone signalling. Endocrinology 143:4856-4867
19 Yamauchi T, Ueki K, Tobe K, Tamemoto H, Sekine N, Wada M, Honjo M, Takahashi M, Takahashi T, Hirai H, Tushima T, Akanuma Y, Fujita T, Komuro I, Yazaki Y, Kadowaki T 1997 Tyrosine phosphorylation of the EGF receptor by the kinase Jak2 is induced by growth hormone. Nature 390:91-96
20 Zhu T, Ling L, Lobie PE 2002 Identification of a JAK2-independent pathway regulating growth hormone (GH)-stimulated p44/42 mitogen-activated protein kinase activity. J Biol Chem 277:46692-45603
21 Shoba L N N, Newman M, Liu W, Lowe WL 2001 LY 294002, an inhibitor of phosphatidylinositol 3-kinase, inhibits GH-mediated expression of the IGF-1 gene in rat hepatocytes. Endocrinology 142:3980-3986
22 Frago L M, Pañeda C, Dickson S L, Hewson A K, Argente J, Chowen J A 2002 Growth hormone (GH) and GH-releasing peptide-6 increase insulin-like growth factor-I expression and activate intracellular signalling pathways involved in neuroprotection. Endocrinology 143:4113-4122
23 Metherell L A, Akker S A, Munroe P B, Rose S J, Caulfield M, Savage M O, Chew S L, Clark A J L 2001 Pseudoexon activation as a novel mechanism for disease resulting in atypical growth-hormone insensitivity. Am J Hum Genet 69:641-646
24 Milward A D, Maamra M, Wilkinson I R, Metherell L A, Savage M O, Clark A J L, Ross R J M 2003 A mutated growth hormone receptor (GHR1-656) with an extracellular domain insertion activates STAT 5 but not MAP kinase. Program of the 85^{th} Annual Meeting of The Endocrine Society, Philadelphia, Pa., 2003, p62 (Abstract)

1-16. (canceled)

17. A method for determining the significance of a given nucleic acid polymorphism or mutation, in a nucleic acid molecule, on the structural properties of a protein encoded by said nucleic acid molecule comprising:
(a) exposing the protein encoded by said nucleic acid molecule to a plurality of proteases;
(b) determining an extent of proteolytic cleavage of said protein; and, optionally,
(c) comparing said extent of proteolytic cleavage of the protein encoded by said nucleic acid molecule with an extent of proteolytic cleavage of a wild-type protein when exposed to said plurality of proteases.

18. A screening method for determining the significance of a plurality of variants of at least one gene comprising:
(a) obtaining a sample of at least one protein variant encoded by the plurality of variants of said at least one gene;
(b) exposing said at least one protein variant to at least one protease;
(c) determining an extent of proteolytic cleavage of said at least one protein variant; and
(d) comparing said extent of proteolytic cleavage of said at least one protein variant with an extent of proteolytic cleavage of a wild-type protein when exposed to the at least one protease.

19. A method according to claim 18 wherein said at least one protein variant is exposed to a plurality of proteases.

20. A method according to claim 19 wherein at least some of said plurality of proteases attack different sites within the at least one protein variant.

21. A method according to claim 20 wherein said plurality of proteases are selected from the group consisting of trypsin, chymotrypsin, proteinase K, aminopeptidase, carboxypeptidase, collagenase, elastase, Kallikrein, metalloendopeptidase, papain, pepsin, and any combination thereof.

22. A method according to claim 18 wherein a plurality of protein variants are exposed to said at least one protease.

23. A method according to claim 22 wherein said plurality of protein variants are exposed to said at least one protease, or vice versa, simultaneously.

24. A method according to claim 22 wherein said plurality of protein variants are exposed to said at least one protease either simultaneously or successively.

25. A method according to claim 22 wherein said plurality of protein variants are exposed to said at least one protease under conditions that support the activity of said at least one protease.

26. A method according to claim 22 wherein digestion of said plurality of protein variants by said at least one protease is terminated by adding at least one protease inhibitor to the reaction.

27. A method according to claim 18 wherein said extent of proteolytic cleavage is determined using a conventional protein assay.

28. A method according to claim 27 wherein said protein assay involves SDS-PAGE analysis.

29. A method according to claim 28 wherein said analysis is followed by staining or blotting.

30. A method according claim 18 wherein additional studies are undertaken to determine the functionality of the at least one protein variant.

31. A method according claim 18 wherein part (d) involves exposing the wild-type protein to said at least one protease, and then determining the extent of proteolytic cleavage of said wild-type protein.
